(19) United States
(12) Patent Application Publication
(10) Pub. No.: US 2014/0273053 A1
(43) Pub. Date: Sep. 18, 2014

(54) CELL CHIPS

(71) Applicants: Solidus BioSciences, Inc., San Francisco, CA (US); Samsung Electro-Mechanics Co. Ltd., Gyunggi-Do (KR)

(72) Inventors: Moo-Yeal Lee, Burlingame, CA (US); Se Hoon Jeong, Seongnam (KR); Bo Sung Ku, Suwon-Si (KR)

(73) Assignees: Samsung Electro-Mechanics Co. Ltd., Gyunggi-Do (KR); Solidus BioSciences, Inc., San Francisco, CA (US)

(21) Appl. No.: 14/179,065

(22) Filed: Feb. 12, 2014

Related U.S. Application Data
(63) Continuation of application No. PCT/US2012/050427, filed on Aug. 10, 2012.
(60) Provisional application No. 61/522,747, filed on Aug. 12, 2011.

(51) Int. Cl. G01N 33/50

(52) U.S. Cl. CPC G01N 33/5014
USPC 435/25; 435/29

(57) ABSTRACT

Disclosed is a cell chip and methods for the use thereof, wherein the cell chip includes a substrate made of an opaque material and having a plurality of insertion holes formed therein, a filler made of a transparent material and inserted into each of the insertion holes so as to protrude from the substrate, and a biomatrix which is formed on the filler and immobilizes a biomaterial. Also, another substrate having a plurality of wells which store a fluid is further provided thus forming a 3D cell chip.

### RELATED APPLICATION

This application is a continuation of International Application No. PCT/US2012/050427, which designated the United States and was filed on Aug. 10, 2012, published in English, which claims the benefit of U.S. Provisional Application No. 61/522,747, filed on Aug. 12, 2011. The entire teachings of the above applications are incorporated herein by reference.

### BACKGROUND OF THE INVENTION

1. Technical Field

The present invention relates to a cell chip.

2. Description of the Related Art

In order to test the toxicity of medicines in vitro, two cell culture methods are typically used One of them is a method of adhering cells to a two-dimensional (2D) surface to culture them (2D cell monolayer culture), and the other is a method of immobilizing cells in a three-dimensional (3D) biomatrix to culture them (3D cell culture). For the 2D cell culture, a microtiter plate (e.g. 6-well, 12-well, 24-well, 96-well, 384-well, 1536-well microtiter plates) comprising a plastic plate and a plurality of wells arranged thereon is used, and cells and a culture broth in an amount approximately ranging from ones of milliliters (ml) to tens of microliters (μl) are added into the wells and thus cultured. The 2D cells which are cultured in such a microtiter plate are advantageous because a variety of simple tests may be rapidly performed at lower cost compared to animal/human clinical tests. For the 3D cell culture, a mixture of cells and hydrogel in an amount of ones of ml to tens of μl is added into the wells and then a culture broth is further added thereon so as to perform culturing. Thus, the cells grow in the 3D biomatrix (e.g., collagen, matrigel, alginate, etc.). Such 3D cells are typically known to be more similar to the bio environment than conventional 2D cells. However, because the cells are immobilized in the wells, it is difficult to wash the cells in the wells after treating them with medicines. This problem may in particular become more serious when the size of the wells is reduced and the number of wells is increased in order that more tests can be performed on a single plate, such as 384-well or 1536-well microtiter plate.

With the goal of solving this problem and performing the test in much smaller volumes, an array-based cell chip in which cells having a fixed 3D structure are immobilized on a flat glass plate subjected to surface treatment and then cultured is described in U.S. Patent Application Publication No. 20090221441, the contents of which are expressly incorporated herein by reference. The test is performed using the array-based cell chip in which collagen or alginate for immobilizing cells are provided in the form of an array on the glass plate, without the formation of the wells. This array-based cell chip is advantageous because it may be more easily washed and its volume is smaller, compared to when conventional microtiter plates are used, and also because a toxicity test of a pharmaceutical may be more rapidly performed. However, there is no physical intercellular partition on the array-based cell chip that has been embodied on the flat plate. Under certain circumstances, this lack of partition allows pharmaceuticals or other test material to undergo cross-merging which can increase the probability of generating a test error and can allow a pharmaceutical that is very small to be exposed to the air and evaporate. In addition, the plate of the cell chip is often made of a transparent material, and thus may cause scattering when the cells are observed using an optical apparatus such as a CCD or the like. Thus, it would be desirable to design a chip that can be used to observe the cells over a long period of time (e.g. one day or longer) and/or using an optical apparatus, even under conditions of high humidity.

### SUMMARY OF THE INVENTION

Accordingly, the present invention is intended to provide a cell chip in which biomatrices for immobilizing a biomaterial are separated from the surface of a substrate by a distance, thus preventing cross-merging between adjacent biomatrices and facilitating a washing process.

Also the present invention is intended to provide a cell chip in which the transparency of a substrate and a filler is adjusted, thus reducing scattering.

Also the present invention is intended to provide a 3D cell chip, which further includes a substrate having wells that store a culture broth or a reagent (which may further include an enzyme, DNA, RNA, an antibody, a virus, etc., as well as the pharmaceutical) supplied to cells, thus creating an environment similar to that of the human body or animal, thereby ensuring accurate and predictable toxicity data.

A first aspect of the present invention provides a cell chip, comprising a substrate made of an opaque material and having a plurality of insertion holes formed therein, a filler made of a transparent material and inserted into each of the insertion holes so as to protrude from the substrate, and a biomatrix which is formed on the filler and immobilizes a biomaterial.

In this aspect, the insertion holes may have a circular or a polygonal cross-sectional shape, and the filler material may have a cross-sectional shape corresponding to the cross-sectional shape of the insertion holes.

In this aspect, the plurality of insertion holes may be provided as an array.

In this aspect, a length of the filler may be greater than a thickness of the substrate.

In this aspect, the biomatrix may be made of a sol-gel comprising extracellular matrix, matrigel, alginate or hydrogel.

In this aspect, the cell chip may further comprise an adhesive layer between the filler and the biomatrix which are in contact with each other.

In this aspect, a plurality of through holes may also be formed from one surface of the substrate to the other surface thereof.

In this aspect, the through holes may have a circular or a polygonal cross-sectional shape.

In this aspect, the through holes may be formed around the insertion holes at positions close thereto.

A second aspect of the present invention provides a cell chip, comprising a lower substrate having a plurality of wells which store a fluid, an upper substrate which is positioned above the lower substrate, wherein said upper substrate is made of an opaque material and has a plurality of insertion holes formed therein, a filler made of a transparent material and inserted into each of the insertion holes so as to protrude from a lower surface of the upper substrate, and a biomatrix, which is formed on the filler, immobilizes a biomaterial, and is inserted into each of the wells.

In this aspect, the insertion holes may have a circular or a polygonal cross-sectional shape, and the filler may have a cross-sectional shape corresponding to the cross-sectional shape of the insertion holes.

In this aspect, the plurality of insertion holes may be provided as an array, and the plurality of wells may correspond to the array of the insertion holes.

In this aspect, a length of the filler may be greater than a thickness of the upper substrate.

In this aspect, the biomatrix may be made of a sol-gel comprising extracellular matrix, matrigel, alginate or hydrogel.

In this aspect, the cell chip may further comprise an adhesive layer between the filler and the biomatrix which are in contact with each other.

In this aspect, a plurality of through holes may also be formed from one surface of the upper substrate to the other surface thereof.

In this aspect, the through holes may have a circular or a polygonal cross-sectional shape.

In this aspect, the through holes may be formed around the insertion holes at positions close thereto.

In this aspect, the through holes may be positioned above the wells so as to communicate with the wells.

In a third aspect, the invention is directed to a method of detecting the effect of a test agent on cells comprising bringing into contact a fluid comprising said test agent a cell chip of the present invention.

### BRIEF DESCRIPTION OF THE DRAWINGS

The features and advantages of the present invention will be more clearly understood from the following detailed description taken in conjunction with the accompanying drawings, in which:

FIG. 1 is a top plane view schematically showing a cell chip according to a first embodiment of the present invention;

FIGS. 2 and 3 are a side view and a cross-sectional view schematically showing the cell chip of FIG. 1, respectively;

FIG. 4 is a cross-sectional view showing a modification of the cell chip of FIG. 3;

FIG. 5 is a side view showing the use of the cell chip of FIG. 1;

FIGS. 6 and 7 are cross-sectional views schematically showing a cell chip according to a second embodiment of the present invention; and

FIG. 8 is a cross-sectional view showing a modification of the cell chip of FIG. 7.

### DETAILED DESCRIPTION OF THE INVENTION

Hereinafter, embodiments of the present invention will be described in detail while referring to the accompanying drawings. The same reference numerals are used throughout the drawings to refer to the same or similar elements. Moreover, descriptions of known techniques, even if pertinent to the present invention, are regarded as unnecessary and may be omitted when they would make the characteristics of the invention and the description unclear.

Furthermore, the terms and words used in the present specification and claims should not be interpreted as being limited to typical meanings or dictionary definitions, but should be interpreted as having meanings and concepts relevant to the technical scope of the present invention based on the rule according to which an inventor can appropriately define the concept implied by the term to best describe the method he or she knows for carrying out the invention.

The words “a” or “an” are meant to encompass one or more, unless otherwise specified.

FIG. 1 is a top plane view schematically showing a cell chip according to a first embodiment of the present invention, FIGS. 2 and 3 are a side view and a cross-sectional view schematically showing the cell chip of FIG. 1, FIG. 4 is a cross-sectional view showing a modification of the cell chip of FIG. 3, and FIG. 5 is a side view showing the use of the cell chip of FIG. 1. With reference thereto, the cell chip according to the present invention is described below.

As shown in FIGS. 1 to 3, the cell chip 100 is configured such that biomatrices 130 for immobilizing a biomaterial C are formed on fillers 120 and the fillers 120 are inserted into the insertion holes 115 of a substrate 110 and protrude from the substrate 110, and thus the biomatrices 130 are separated from the surface of the substrate 110 by a distance.

The substrate 110 is made of an opaque material. A non-limiting example thereof includes a plastic substrate, a ceramic substrate or the like. Alternatively, the substrate 110 is formed using a modified material that has been obtained by adding a dye to a transparent material or attaching a piece of foamable tape to the surface of a transparent material and then applying a colored paint thereon by means of CVD (Chemical Vapor Deposition) thus decreasing the light transmittance. The shape of such a substrate 110 is not limited, and the thickness thereof may be adjusted appropriately.

The plurality of insertion holes 115 is formed in the substrate 110. The insertion holes 115 can have a variety of shapes, including, for example, a circular shape or a polygonal shape. The shape of the insertions holes can be adapted to increase the contact surface so as to enhance the bondability when the fillers 120 which will be described later are inserted and fixed therein.

The fillers 120 on which the biomatrices 130 are formed are respectively inserted into the plurality of insertion holes 115, so that a plurality of unit cell chips (each including a filler and a biomatrix having an immobilized biomaterial) is formed on a single substrate 110. In order to more easily embody the classification and comparison of the unit cell chips, the unit cell chips may be provided as an array. The array of such unit cell chips results from forming an array of the plurality of insertion holes 115. Although a 4×7 array is depicted in FIG. 1, it is merely illustrative, and the number of unit cell chips may be changed.

The fillers 120 are inserted into the insertion holes 115 and fixed to protrude from the substrate 110. The biomatrices 130 are formed on one surface of the fillers 120, as will be described later, and the fillers 120 function to separate the biomatrices 130 from the surface of the substrate 110 by a distance. When the biomatrices 130 and the substrate 110 are separated in this way, cross-merging between the adjacent biomatrices 130 may be decreased or prevented when a reagent is supplied to the biomatrices 130.

When the fillers 120 are inserted into the insertion holes 115, they are fixed by a predetermined amount of the force of friction occurring between the outer surface of the fillers 120 and the inner wall of the insertion holes 115. Accordingly, the cross-sectional shape of the fillers 120 corresponds to the cross-sectional shape of the insertion holes 115. In particular, when the fillers 120 are forcibly inserted by external pressure, the bondability of fillers 120 to the substrate 110 may be enhanced. However, there is no essential need to forcibly insert the fillers 120 into the insertion holes 115, and a double injection process may, for example, be employed, which includes forming a substrate 110 including insertion holes 115 and then filling the insertion holes 115 with a resin thus forming fillers 120.

The fillers 120 which are inserted into the insertion holes 115 and fixed to the substrate 110 are movably fixed, and thus the fillers 120 be replaced, as needed. A conventional cell chip is typically provided in disposable form because the biomaterial is attached to the cell chip thus making it difficult to be washed. However, in the present invention, the fillers 120 may be replaced thus solving the washing problem, and part of the fillers 120 may be detached from the substrate 110 in order to change the test conditions of specific unit cell chips during the test procedure.

Furthermore, because the fillers 120 are movable, the distance between the biomatrices 130 and the substrate 110, which are separated from each other, may be adjusted per unit cell chip, as needed.

In the case where the plurality of fillers 120 is respectively inserted into the insertion holes 115, in order to separate the biomatrices 130 by the same height from the substrate 110, the fillers 120 may be formed to have the same length, and this length of the fillers 120 may be greater than the thickness of the substrate 110.

In the insertion of the fillers 120 into the insertion holes 115, as shown in FIG. 3, when the fillers 120 are completely inserted into the insertion holes 115 and thus the other surface of the fillers 120 is flush with the outer surface of the substrate 110, the center of gravity of the cell chip 100 is decreased and the cell chip becomes stable. In order to satisfy such conditions and separate the biomatrices 130, which was formed on one surface of the fillers 120, from the surface of the substrate 110 by a distance, the length of the fillers 120 should be greater than the thickness of the substrate 110.

The fillers 120 are made of a transparent material, unlike the substrate 110. For example, glass or transparent plastic may be used In addition, any material which is known may be used for the fillers 120 as long as it is transparent.

In the present invention, the substrate 110 is made of an opaque material and the fillers 120 are made of a transparent material, whereby it is easy to observe the biomaterial C during or after the test. A conventional cell chip is problematic because it is made of an opaque material and thus the biomaterial should be removed from the cell chip so as to be observed, and another conventional cell chip is disadvantageous because it is made exclusively of a transparent material and thus scattering may occur upon use of an optical apparatus such as a CCD, undesirably making it difficult to precisely observe the biomaterial C.

In the present invention, the fillers 120 on which the biomaterial C is disposed are made of a transparent material, unlike the substrate 110. Even when using an optical apparatus, scattering may be decreased, thus facilitating the observation of the biomaterial C.

The biomatrices 130 formed on the fillers 120 function to immobilize the biomaterial C, and the term “biomaterial” refers to any of a variety of biomolecules. Examples of the biomolecules include cells and can also include nucleic acids (e.g., DNA, RNA, oligonucleotides, cDNA, extranuclear genes (plasmids), etc.), peptides, proteins, fats, protein or lipid membranes, organic or inorganic chemical molecules (e.g., pharmaceutical or other compounds), virus particles, blast cell components or cell organelles. The cells can, for example, be eukaryotic cells or prokaryotic cells. Cells that can be as a biomaterial, or the tissues/organs the cells can be derived from, include, but are not limited to, bone marrow, skin, cartilage, tendon, bone, muscle (including cardiac muscle), blood vessels, corneal, neural, brain, gastrointestinal, renal, liver, pancreatic (including islet cells), lung, pituitary, thyroid, adrenal, lymphatic, salivary, ovarian, testicular, cervical, bladder, endometrial, prostate, vulval, esophageal, etc. Also included are the various cells of the immune system, such as T lymphocytes, B lymphocytes, polymorphonuclear leukocytes, macrophages, and dendritic cells.

The biomatrices 130 may be made of a sol-gel, an inorganic material, an organic polymer, or an organic-inorganic composite, which is able to immobilize the biomaterial. In particular, the biomatrices 130 may include an extracellular matrix such as collagen which is porous so that a fluid is moved by diffusion, and a hydrogel such as matrigel or alginate which is non-toxic to the biomaterial.

Such biomatrices 130 enable the fluid to be supplied to the biomaterial C by diffusion, so that an environment similar to the bio environment is furnished to the biomaterial C, or an environment adapted for a specific test is formed.

The biomatrices 130 for immobilizing the biomaterial C are formed by spotting a mixture of the biomaterial C and the biomatrices 130 on one surface of the fillers 120, or may be formed by spotting biomatrices 130 and then spotting the biomaterial C thereon. In particular, in the case where the biomatrices 130 are formed by spotting the mixture of biomaterial C and biomatrices 130 on the fillers 120, the biomaterial C is embedded in the biomatrices 130.

In order to enhance the bondability between the fillers 120 and the biomatrices 130, an adhesive layer (not shown) may be further formed between the fillers 120 and the biomatrices 130, which are in contact with each other. The adhesive layer may be applied to one surface of the fillers 120 and then the biomatrices 130 may be formed thereon using spotting.

For example, in the case where alginate is used for the biomatrices 130, the adhesive layer may be composed of a mixture of poly-L-lysine (PLL)-barium chloride.

Also as shown in FIG. 4, a plurality of through holes 140 may be formed from one surface of the substrate 110 to the other surface thereof so as to perforate the substrate 110. When warping occurs from one side of the substrate 110 to the other side thereof; it may cause a test error. However, when the through holes 140 are formed in this way, warping may be stopped from occurring and thus the warping of the entire substrate 110 may be reduced. In order to enhance the effect of stopping the warping, the through holes 140 may be repetitively formed at a predetermined interval in a width direction or a length direction. Specifically, the through holes 140 may be formed around the insertion holes 115 at positions close thereto. The cross-sectional shape of the through holes 140 is not particularly limited, but may be circular or polygonal.

The cell chip 100 according to the present embodiment enables the reaction for a specific reagent to be observed by supplying the reagent to the biomaterial C. As shown in FIG. 5, the reagent is directly supplied to the biomatrices 130 using a supplier such as a pipette D. The plurality of fillers 120 is bound on a single substrate 110 and the plurality of biomatrices 130 is disposed thereon, thus making it possible to simultaneously observe the changes in the biomaterial C in response to various reagents.

FIGS. 6 and 7 are cross-sectional views schematically showing a cell chip according to a second embodiment of the present invention. With reference thereto, the cell chip according to the present embodiment is described below.

The cell chip 200 according to the present embodiment further includes a lower substrate 210 having a plurality of wells 220 that store at least one (hereinafter, referred to as a fluid) among a reagent and a culture broth to be supplied to the cell chip as shown in FIGS. 1 to 3. Below, an upper substrate 110′, fillers 120′, and biomatrices 130′ may be configured corresponding to the configuration as described with reference to FIGS. 1 to 3, and thus the detailed description thereof is omitted.

As shown in FIG. 6, the upper substrate 110′ has a plurality of insertion holes 115′, and the fillers 120′ are inserted into the insertion holes 115′. As such, the fillers 120′ are inserted to protrude from the lower surface of the upper substrate 110′, and the biomatrices 130′ for immobilizing the biomaterial C are inserted into the wells 220 of the lower substrate 210 which will be described below and thus formed on the lower surface of the fillers 120′.

The lower substrate 210 is disposed under the upper substrate 110′ so that the fluid F is supplied to the biomaterial. The lower substrate 210 may include a glass plate, a plastic plate, a metal plate or a ceramic plate, and has the plurality of wells 220.

The fluid F which is stored in the wells 220 may include not only a reagent or a culture broth necessary for a specific test in order to furnish an environment more similar to the bio environment to the biomaterial C, but also a dyeing material (e.g., fluorescent and luminous materials), protein, plasmids, DNA, interference RNA, antigen/antibody, viruses, etc., which are to be analyzed.

FIG. 7 shows the upper substrate 110′ and the lower substrate 210, which are connected with each other.

The biomatrices 130′ for immobilizing the biomaterial C are inserted into the wells 220, and the fluid F is supplied to the biomatrices 130′ and moves to the biomaterial C under the influence of diffusion. The shape of the wells 220 is not limited, but the area of the wells 220 is larger than that of the biomatrices 130′ so that the biomatrices 130′ may be inserted therein, and the depth thereof is also larger than the height of the biomatrices 130′.

The upper substrate 110′ and the lower substrate 210 may be functionally separated, thus solving the washing problem of the conventional microtiter plate and also solving cross-merging or drying problems of the conventional array-based cell chip.

When the upper substrate 110′ and the lower substrate 210 are connected with each other, as shown in FIG. 7, they may be connected so as to come into contact with each other. However, in order to efficiently supply air, the upper substrate 110′ and the lower substrate 210 may be connected with each other so that they are separated by a distance (in such a manner that a protrusion member may be interposed between the upper substrate 110′ and the lower substrate 210). In this case, the distance between the upper substrate 110′ and the lower substrate 210, which are separated from each other, may vary within the range in which the biomatrices 130′ are inserted into the wells 220.

In the case where the insertion holes 115′ are provided as an array, the plurality of biomatrices 130′ may also be provided as an array. Thus, the wells 220 may have an array corresponding to the insertion holes 115′ so that the biomatrices 130′ may be inserted therein.

In the cell chip 200, even when the plurality of biomatrices 130′ for immobilizing the same biomaterial C is formed on the upper substrate 110′, changes in the biomaterial C may be observed under various conditions using the different fluid F (in particular, the different reagent). Even when the biomatrices 130′ for immobilizing a variety of biomaterials C are formed on the upper substrate 110′, changes in the biomaterial C may be observed under the same environment using the same fluid F.

FIG. 8 is a cross-sectional view showing a modification of the cell chip of FIG. 7. As shown in FIG. 8, a plurality of through holes 140′ may be formed from one surface of the upper substrate 110′ to the other surface thereof so as to perforate the upper substrate 110′. When warping occurs from one side of the upper substrate 110′ to the other side thereof; the upper substrate 110′ and the lower substrate 210 may not be matched properly, undesirably causing a test error. When the through holes 140′ are formed, warping may be stopped, thus reducing warping of the entire substrate 110′. In order to increase the effect of stopping the warping, the through holes 140′ may be repetitively formed at a predetermined interval in a width direction or a length direction. Specifically, the through holes 140′ may be formed around the insertion holes 140′ at positions close thereto. More specifically, the through holes 140′ are positioned above the wells 220 so as to communicate with the wells 220, and thereby may be utilized as a discharge path of bubbles generated upon reaction of the biomaterial C with a reagent. When this structure is employed, it is easy to remove bubbles, and the inflow of air becomes much easier. The cross-sectional shape of the through holes 140′ is not particularly limited, but may be circular or polygonal.

As discussed above, the invention also encompasses methods of using the cell chips described herein, for example, to detect the effect of a test agent on the cells and a test agent. In one embodiment, the invention is directed to a method of detecting a reaction between cells and a test agent comprising bringing into contact a fluid comprising said test agent and an upper substrate, which is positioned above the lower substrate, is made of an opaque material and has a plurality of insertion holes formed therein;

wherein a filler made of a transparent material is inserted into each of the insertion holes so as to protrude from a lower surface of the upper substrate; and wherein a biomatrix, which is formed on the filler, immobilizes said cells, and is inserted into each of the wells, wherein said fluid and said test agent are brought into contact for a time sufficient for an interaction between the cells and the test agent, said method further comprising detecting the effect of said test agent on said cells.

As will be understood, in certain embodiments, the test agent can comprise an agent capable of interacting with the biomaterial. In certain aspects, the test agent within the micro-wells can be the same or can be different and can comprise for, example, cytochrome P450, a pharmaceutical, a cosmetic, a cosmeceutical, a small molecule drug, a biopolymer or a combination thereof. In additional embodiment, the reagent in the micro-wells comprises a matrix, micromatrix or biomatrix containing cytochrome p450 is deposited within the well surface of the lower substrate. Common P450 isoforms that are applicable are 1A1, 1A2, 1B1, 2A6, 2B6, 2C8, 2C9, 2C18, 2C19, 2D6, 2E1, 2J2, 3A4, 3A5, 3A7, 4B1, 4F8, 4F12, 7B1, 26B1, 27A1, and 39A1. In addition to P450s, other Phase I metabolism-based enzymes can be used, including flavin monooxygenases, monoamine oxidases, various esterases, quinone reductases, peroxidases, and alcohol dehydrogenases. In addition to Phase I enzymes, Phase II metabolism-based enzymes can be used, including uridinyl glucuronosyl transferases (particularly isoforms 1A1, 1A3, 1A4, 1A5, 1A6, 1A7, 1A8, 1A9, 1A10, 2B4, 2B7, 2B10, 2B11, 2B15, ad 2B17), epoxide hydrolases, N-acetyl transferases, glutathione S-transferases, sulfotransferases (particularly isoforms 1A1, 2B1a, 2B1b, and 1E1), and catechol O-methyltransferases. In addition to the aforementioned enzymes and their isoforms, a wide range of synthetically relevant enzymes from human and non-human sources can be used, including those contained within Enzyme Commission (EC) Classes 1-6, e.g., Class 1 (oxidoreductases), Class 2 (transferases), Class 3 (hydrolases), Class 4 (lyases), Class 5 (isomerases), and Class 6 (ligases). The microwells can also for example, comprise culture broth necessary for cell growth and a labeling material such as fluorescent label, dyes and luminous materials.

The effect on the cells that can be studied include, for example, detecting cell proliferation, cell death (cytotoxicity), and other metabolic or morphological changes in cells.

As described hereinbefore, the present invention provides a cell chip. According to the present invention, the cell chip is advantageous because a culture broth and a reagent can be supplied under the influence of diffusion to a biomaterial immobilized to the biomatrices, thus making an environment similar to the bio environment, and also because the biomaterial is separated from the substrate by a distance, thus preventing mixing or cross-merging problems between adjacent biomaterials.

Also according to the present invention, the transparency of the substrate and the fillers is adjusted, thus reducing scattering upon observation of the biomaterial using an optical apparatus, thereby facilitating the observation of the biomaterial.

Also according to the present invention, a lower substrate for supplying a culture broth or a reagent and an upper substrate having biomatrices formed thereon are functionally and spatially separated from each other, thus preventing the mixing or cross-merging problems between adjacent biomaterials, thereby obtaining a 3D cell chip for making an environment similar to that of the human body or animal.

Although the embodiments of the present invention have been disclosed for illustrative purposes, those skilled in the art will appreciate that a variety of different modifications, additions and substitutions are possible, without departing from the scope and spirit of the invention as disclosed in the accompanying claims. Accordingly, such modifications, additions and substitutions should also be understood as falling within the scope of the present invention.

While the present invention has been shown and described in connection with the exemplary embodiments, it will be apparent to those skilled in the art that modification and variation can be made withough departing from the spirit and scope of the invention as defined by the appended claims.

1. A cell chip comprising:
a substrate made of an opaque material and having a plurality of insertion holes formed therein;
a filler made of a transparent material and inserted into each of the insertion holes so as to protrude from the substrate; and
a biomatrix which is formed on the filler and immobilizes a biomaterial.

2. The cell chip of claim 1, wherein the insertion holes have a circular or a polygonal cross-sectional shape, and the filler has a cross-sectional shape corresponding to the cross-sectional shape of the insertion holes.

3. The cell chip of claim 1, wherein the plurality of insertion holes is provided as an array.

4. The cell chip of claim 1, wherein the length of the filler is greater than a thickness of the substrate.

5. The cell chip of claim 1, wherein the biomatrix is made of a sol-gel comprising extracellular matrix, matrigel, alginate or hydrogel.

6. The cell chip of claim 1, further comprising an adhesive layer between the filler and the biomatrix which are in contact with each other.

7. The cell chip of claim 1, wherein a plurality of through holes is formed from one surface of the substrate to the other surface thereof.

8. The cell chip of claim 7, wherein the through holes have a circular or a polygonal cross-sectional shape.

9. The cell chip of claim 7, wherein the through holes are formed around the insertion holes at positions close thereto.

10. The cell chip of claim 1, wherein the biomaterial is cells.

11. A cell chip, comprising:
a lower substrate having a plurality of wells which store a fluid;
an upper substrate, which is positioned above the lower substrate, is made of an opaque material and has a plurality of insertion holes formed therein;
a filler made of a transparent material and inserted into each of the insertion holes so as to protrude from a lower surface of the upper substrate; and
a biomatrix, which is formed on the filler, immobilizes a biomaterial, and is inserted into each of the wells.

12. The cell chip of claim 11, wherein the insertion holes have a circular or a polygonal cross-sectional shape, and the filler has a cross-sectional shape corresponding to the cross-sectional shape of the insertion holes.

13. The cell chip of claim 11, wherein the plurality of insertion holes is provided as an array, and the plurality of wells correspond to the array of the insertion holes.

14. The cell chip of claim 11, wherein a length of the filler is greater than a thickness of the upper substrate.

15. The cell chip of claim 11, wherein the biomatrix is made of a sol-gel comprising extracellular matrix, matrigel, alginate or hydrogel.

16. The cell chip of claim 11, further comprising an adhesive layer between the filler and the biomatrix which are in contact with each other.

17. The cell chip of claim 11, wherein a plurality of through holes is formed from one surface of the upper substrate to the other surface thereof.

18. The cell chip of claim 17, wherein the through holes have a circular or a polygonal cross-sectional shape.

19. The cell chip of claim 17, wherein the through holes are formed around the insertion holes at positions close thereto.

20. The cell chip of claim 16, wherein the through holes are positioned above the wells so as to communicate with the wells.

21. The cell chip of claim 11, wherein the biomaterial is cells.

22. A method of detecting the effect of a test agent on cells comprising bringing into contact a fluid comprising said test agent and a cell chip of claim 1.

23. A method of detecting the effect of a test agent on cells comprising using the cell chip of claim 11.

24. A method of detecting the effect of a test agent on cells comprising bringing into contact a fluid comprising said test agent and an upper substrate, which is positioned above the lower substrate, is made of an opaque material and has a plurality of insertion holes formed therein;
wherein a filler made of a transparent material is inserted into each of the insertion holes so as to protrude from a lower surface of the upper substrate; and wherein a biomatrix, which is formed on the filler, immobilizes said cells, and is inserted into each of the wells, wherein said fluid and said test agent are brought into contact for a time sufficient for reaction between the cells and the test agent, said method further comprising detecting the effect of the test agent on the said cells.

25. The method of claim 24, wherein the fluid that comprises the test agent is in a plurality of cells of a second substrate.

26. The method of claim 24, wherein the test agent is selected from the group consisting an enzyme, a small molecule, a biopolymer or a combination thereof.

27. The method of claim 24, wherein the enzyme is a cytochrome P450 enzyme.
